(19) United States
(12) Patent Application Publication
(10) Pub. No.: US 2026/0306826 A1
(43) Pub. Date: Oct. 8, 2026

(54) Skin Care Device

(71) Applicant: Meditherapy Co., Ltd., Seoul (KR)

(72) Inventors: Seung Jin Lee, Yongin-si (KR); Han Seung Lee, Seoul (KR)

(73) Assignee: Meditherapy Co., Ltd., Seoul (KR)

(21) Appl. No.: 19/449,323

(22) Filed: Jan. 14, 2026

(30) Foreign Application Priority Data
Apr. 2, 2025 (KR) 10-2025-0042987

(51) Int. Cl. A61N 1/04; A61N 5/06

(52) U.S. Cl. CPC A61N 1/042; A61N 1/0476; A61N 5/0616; A61N 2005/0644; A61N 2005/0651; A61N 2005/0659; A61N 2005/0663

(57) ABSTRACT

A skin device according to an embodiment disclosed herein includes a body, a head part disposed at one end of the body, and a receiver disposed in an internal space of the body, wherein the head part transmits electrical stimulation, light irradiation, and vibration to a user's skin, wherein the body includes a main body part having a storage space, and a button, wherein the receiver includes a receiving body having a receiving space therein, wherein the receiving body forms a groove, wherein the head part includes a first layer part and a second layer part, wherein the first layer part is seated and coupled to the second layer part, wherein the first layer part includes a first electrode and a second electrode, and wherein each of the first electrode and the second electrode has a circular rim shape.

### CROSS-REFERENCE TO RELATED APPLICATION

The present application claims the right of priority to and the benefits of Korean Application No. 10-2025-0042987 having a filing date of Apr. 2, 2025, the content of which is hereby incorporated by reference in its entirety.

### TECHNICAL FIELD

The present invention relates to a skin device.

### BACKGROUND OF THE INVENTION

Skin devices generally consist of a combination of a main body and a handpiece, include multiple functions, and are mostly used through professional procedures. Representative functions constituting such a system may include UltraSonic, Radio Frequency (RF), Iontophoresis, High-Intensity Focused Ultrasound (HIFU), Electroporation, CRYOTHERAPY, and the like.

Such skin devices have a disadvantage in that professional treatment is required due to high product cost, installation requirements, complexity of use, and portability issues.

Recently, home beauty devices have been released, but they still have a high unit price and the inconvenience of being used while connected to a power source.

### SUMMARY OF THE INVENTION

An object of the present invention is to provide a skin device that can be used wirelessly by charging and can be easily used by being coupled to a cosmetic container or the like.

The objects of the present invention are not limited to the objects mentioned above, and other technical objects not mentioned will be clearly understood by those skilled in the art from the description below.

A skin device according to an embodiment for solving the above problem includes a body and a head part disposed at one end of the body, wherein the head part transmits electrical stimulation, light irradiation, and vibration to a user's skin.

The body may include a main body part including a storage space capable of accommodating components therein, and a button disposed on an outer surface of the main body part to control power.

The body may further include a grip, and the grip may be formed on the outer surface of the main body part and disposed in a form recessed inward from the outer surface of the main body part.

The skin device may further include a receiver disposed in the internal space of the body, wherein the receiver includes a receiving body including a receiving space therein, and the receiving body may include a groove.

The head part may include a first region configured to irradiate light and transmit vibration, a second region configured to transmit electrical stimulation and vibration, a third region configured to irradiate light and transmit vibration, a fourth region configured to transmit electrical stimulation and vibration, and a fifth region configured to irradiate light and transmit vibration.

An area of the first region may be smaller than an area of the second region, the area of the second region may be smaller than an area of the third region, the area of the third region may be smaller than an area of the fourth region, and the area of the fourth region may be larger than an area of the fifth region.

The first region may have a circular rim shape surrounding the outside of the groove, the second region may have a circular rim shape surrounding the outside of the first region, the third region may have a circular rim shape surrounding the outside of the second region, the fourth region may have a circular rim shape surrounding the outside of the third region, and the fifth region may have a circular rim shape surrounding the outside of the fourth region.

The head part may include a first layer part and a second layer part, and the first layer part may be seated and coupled to the second layer part.

The first layer part may include electrodes for transmitting electricity, and the electrodes may include a first electrode and a second electrode.

Each of the first electrode and the second electrode may have a circular rim shape.

A diameter of the first electrode may be smaller than a diameter of the second electrode.

The second layer part may be coupled to a lower portion of the main body part, the second layer part may include a through-hole, and the groove may be exposed through the through-hole.

The second layer part may output light to the outside and transmit vibration.

The second layer part may include a first seating part and a second seating part, and each of the first seating part and the second seating part may have a circular rim shape.

The first seating part and the second seating part may be spaced apart from each other in a first direction.

The first electrode may be seated on the first seating part, and the second electrode may be seated on the second seating part.

The first electrode and the second electrode may transmit electrical stimulation to the user's skin.

Each of the first electrode and the second electrode may be made of a SUS (stainless steel) material.

A lower surface of the second layer part may be made of a transparent or translucent material.

The groove may include a first groove and a second groove, the first groove and the second groove may be sequentially positioned in a second direction crossing the first direction, and a diameter of the first groove in the first direction may be smaller than a diameter of the second groove in the first direction.

The skin device may further include a receiver disposed in the internal space of the body, and may be configured such that a cosmetic container is coupled to and separated from the receiver.

Specific details of other embodiments are included in the detailed description and drawings.

According to the skin device according to an embodiment, it can be easily used by being connectable to and detachable from a cosmetic container, and at the same time, after applying a cosmetic composition to a user's face, the skin absorption rate of the cosmetic composition can be increased by using the device for a cosmetic container.

The effects according to the embodiments are not limited by the contents exemplified above, and more diverse effects are included in the present specification.

### BRIEF DESCRIPTION OF THE DRAWINGS

FIGS. 1 and 2 are views schematically illustrating a skin device according to an embodiment.

FIG. 3 is a view schematically illustrating a front side of a skin device according to an embodiment.

FIG. 4 is a view schematically illustrating a rear side of a skin device according to an embodiment.

FIG. 5 is a view schematically illustrating a side surface of a skin device according to an embodiment.

FIG. 6 is a view schematically illustrating a lower surface of a skin device according to an embodiment.

FIG. 7 is a view schematically illustrating an exploded state of a skin device according to an embodiment.

FIG. 8 is a view schematically illustrating a second layer part of a skin device according to an embodiment.

FIG. 9 is a view schematically illustrating a first layer part of a skin device according to an embodiment.

FIGS. 10 and 11 are views schematically illustrating a receiver of a skin device according to an embodiment.

### DETAILED DESCRIPTION OF THE INVENTION

Advantages and features of the present invention and methods for achieving them will become clear with reference to the embodiments described below in detail together with the accompanying drawings. However, the present invention is not limited to the embodiments disclosed below but will be implemented in various different forms; these embodiments are provided only to make the disclosure of the present invention complete and to fully inform those of ordinary skill in the art of the scope of the invention, and the present invention is only defined by the scope of the claims.

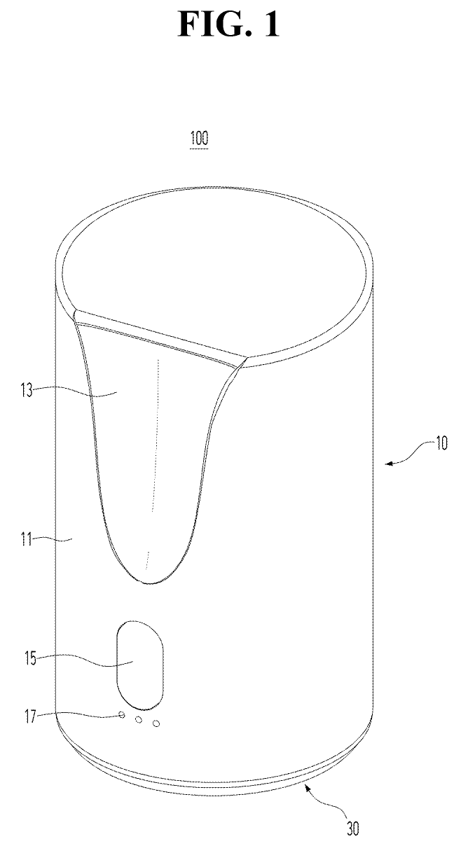

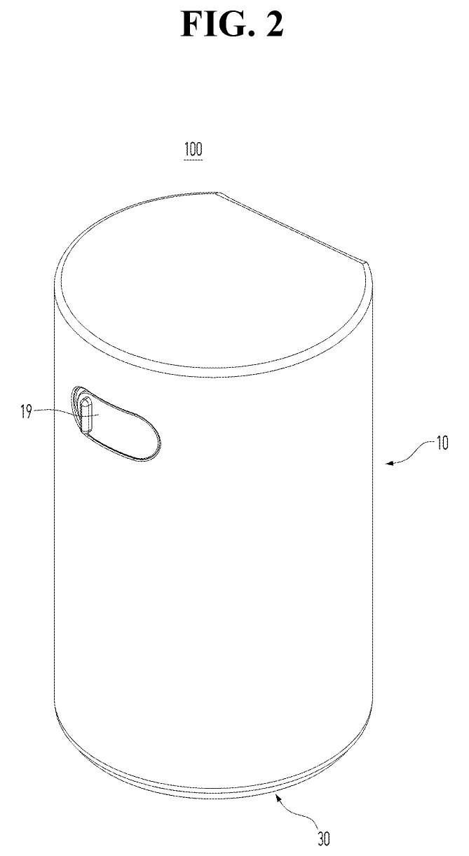

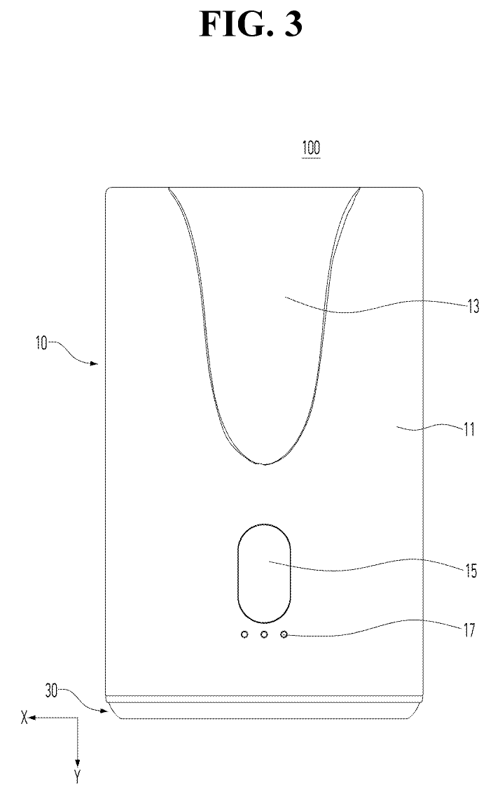

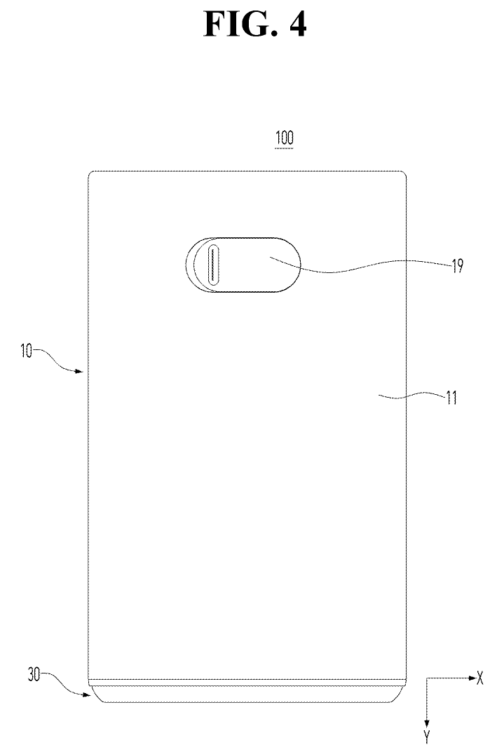

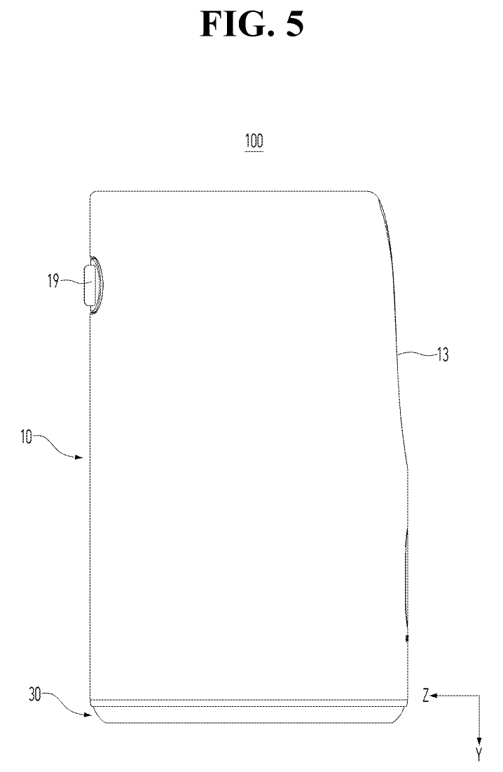

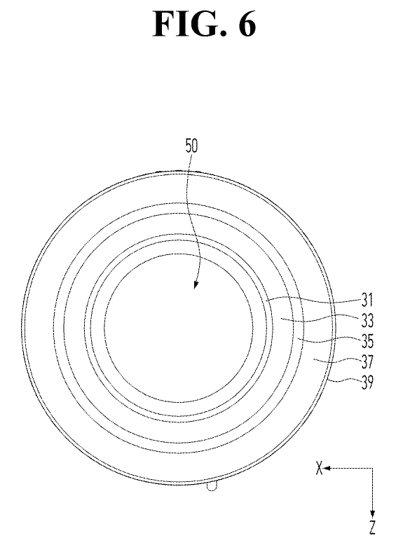

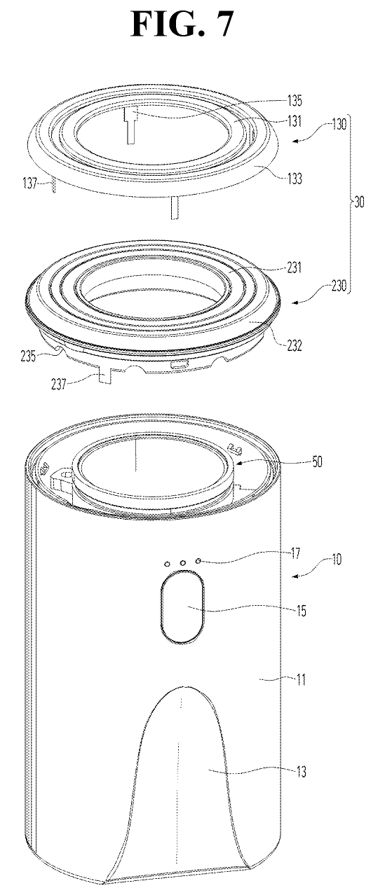

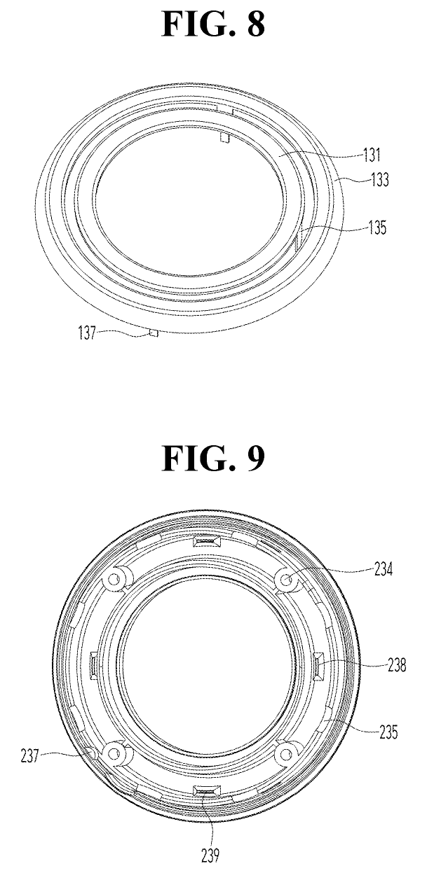

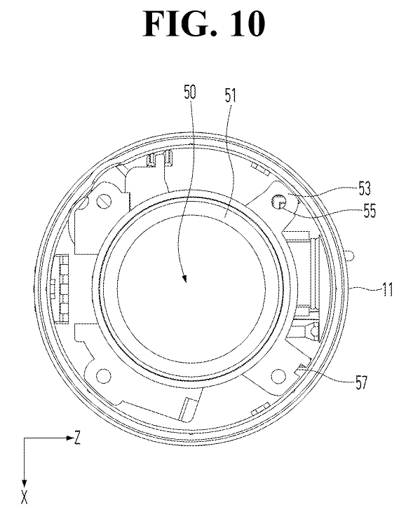

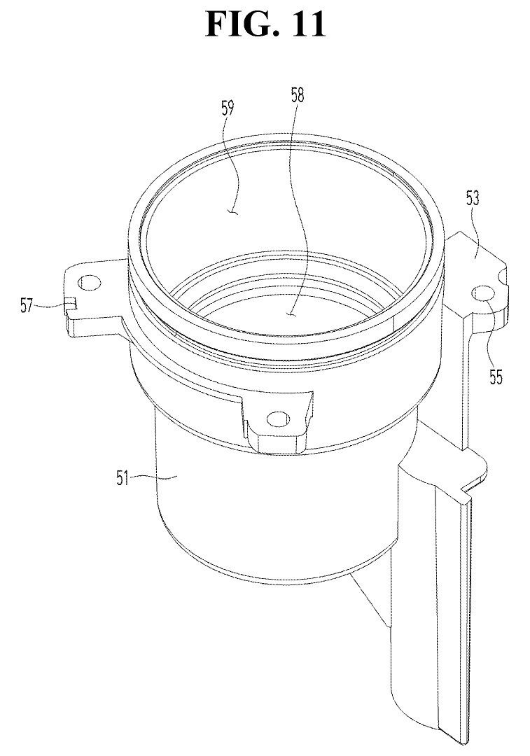

FIGS. 1 and 2 are views schematically illustrating a skin device according to an embodiment, FIG. 3 is a view schematically illustrating a front side of a skin device according to an embodiment, FIG. 4 is a view schematically illustrating a rear side of a skin device according to an embodiment, FIG. 5 is a view schematically illustrating a side surface of a skin device according to an embodiment, FIG. 6 is a view schematically illustrating a lower surface of a skin device according to an embodiment, FIG. 7 is a view schematically illustrating an exploded state of a skin device according to an embodiment, FIG. 8 is a view schematically illustrating a second layer part of a skin device according to an embodiment, FIG. 9 is a view schematically illustrating a first layer part of a skin device according to an embodiment, and FIGS. 10 and 11 are views schematically illustrating a receiver of a skin device according to an embodiment.

Referring to FIGS. 1 and 2, a skin device 100 according to an embodiment may include a body 10 and a head part 30 disposed at one end of the body 10. In addition, the skin device 100 may further include a receiver 50 disposed inside the body 10.

In one embodiment, the receiver 50 may pass through the body 10 and be recessed toward the inside of the body 10. A part of a cosmetic container may be inserted and fixed into the receiver 50.

As such, the skin device 100 according to an embodiment can be easily used as it is connectable to and detachable from a cosmetic container, and at the same time, after applying a cosmetic composition to a user's face, the skin absorption rate of the cosmetic composition can be increased by using the device for a cosmetic container. In addition, the skin device 100 according to an embodiment may be a component of a cosmetic container as a cap that is coupled to and separated from the cosmetic container.

The head part 30 of the skin device 100 is in close contact with the facial skin of the user and is equipped with complex functions of electrical stimulation for electroporation, light irradiation, and vibration to effectively assist skin absorption of a cosmetic composition. For example, the head part 30 transmits electrical stimulation to the skin to form an electric field in the cell membrane of the skin to create temporary pores, so that the active ingredients of the cosmetic composition can be injected deep into the skin. Furthermore, since the head part 30 has a vibration function, it effectively helps absorption of active ingredients of the cosmetic composition. In addition, the head part 30 can induce skin activation of the user by outputting light. In one embodiment, the light may include red light (light in a wavelength range of 620 nm to 750 nm) that promotes skin cell regeneration and collagen production, near-infrared light (light in a wavelength range of 750 nm to 1200 nm) that penetrates deep into the skin to promote collagen and elastin production, blue light (light in a wavelength range of 405 nm to 470 nm) that sterilizes acne-causing bacteria and regulates sebum, and green light (light in a wavelength range of 520 nm to 560 nm) that alleviates pigmentation and has a skin-soothing effect. In another embodiment, the light may include light having two or more wavelength ranges. For example, at least two or more of red light, near-infrared light, blue light, and green light may be output.

To this end, the skin device 100 may include components such as an LED module for irradiating light, a vibration motor for generating vibration, electrodes for contacting the skin to transmit electricity, and a built-in battery. In addition, it may include a pulse circuit for generating a pulse, a voltage amplification circuit for boosting the battery voltage to a high voltage, a control MCU for controlling pulse cycle/intensity/interval, an LED driver for supplying a stable current to the LED module, and a control circuit for ON/OFF, timer, and intensity adjustment. Such components are built inside the body 10 to perform functions of electrical stimulation, light irradiation, and vibration transmission to the user's skin through the head part 30.

The body 10 may include a main body part 11, a grip 13, a button 15, a display 17, and a charging terminal 19.

The main body part 11 may be formed in a cylindrical shape and include a storage space for accommodating components therein. However, it is not limited thereto, and the main body part 11 may be formed in various shapes such as polygonal prism shapes (triangular prism, quadrangular prism, etc.), an elliptical shape, a spherical shape, and the like, and convex portions and concave portions may be formed on the outer surface of the main body part 11 for a user's grip.

In one embodiment, the grip 13 is formed on the outer surface of the main body part 11 and may be disposed in a form recessed inward from the outer surface of the main body part. In addition, the grip 13 may be in a recessed form disposed to correspond to a thumb. Such a grip 13 increases the user's grip feeling as described above and has an anti-slip effect. In another embodiment, an anti-slip pattern may be disposed on the grip 13. The anti-slip pattern may have a shape protruding in a semi-circular shape, and a plurality of anti-slip patterns may be disposed spaced apart on the outer surface of the grip 13. The anti-slip pattern may be made of a silicone material, a urethane material, or a rubber material, but is not limited thereto.

The button 15 is disposed on the outer surface of the main body part 11 and is connected to a control circuit to control the operation of the skin device 100. In one embodiment, the button 15 may control functions such as power ON/OFF and intensity adjustment of 1 to 3 levels, but is not limited thereto.

The display 17 may display operation and output stages. The display may include a lamp that outputs three lights, but is not limited thereto.

The charging terminal 19 is a charging port through an external power source and is connected to a built-in battery to charge the built-in battery from the external power source. The charging terminal 19 may include a charging port and a port cover covering the same.

Referring to FIGS. 3-5, the main body part 11 of the body 10 may include an upper surface extending in a first direction (X-axis) and an outer surface extending in a second direction (Y-axis direction) (a vertical direction) crossing the first direction (X-axis direction). The outer surface may be a surface having a certain curvature, but is not limited thereto.

The grip 13 may be in a form that extends from an end of the upper surface of the main body part 11 (Note: main body part 110 in drawing labels) in the second direction (Y-axis direction) and is recessed. The grip 13 may be in a form recessed in a third direction (Z-axis direction) intersecting each of the first direction (X-axis direction) and the second direction (Y-axis direction) in a shape corresponding to a finger.

The button 15 is disposed on the outer surface of the main body part 110 but may be disposed spaced apart from the grip 13 in the second direction (Y-axis direction). The button 15 may be formed in a bar (BAR) shape extending in the second direction (Y-axis direction), but both ends thereof may be rounded. However, it is not limited thereto and may be formed in various shapes.

The display 17 may include a plurality of lamps disposed spaced apart in the first direction (X-axis direction), and light may be output to the lamps according to operation. The display 17 is disposed on the outer surface of the main body part 110 but may be disposed spaced apart from the button 15 in the second direction (Y-axis direction).

The charging terminal 19 includes a charging port and a port cover covering the same, and may be disposed on the outer surface of the main body part 110. The port cover may be formed in a bar (BAR) shape extending in the first direction (X-axis direction), but both ends thereof may be rounded. However, it is not limited thereto and may be formed in various shapes.

Referring to FIGS. 6-11, the receiver 50 is disposed in the storage space inside the main body part 11 along with the aforementioned components, and the head part 30 may be disposed below the main body part 11. The receiver 50 may include a groove, and the head part 30 may be disposed below the main body part 11 in a form surrounding the outside of the groove of the receiver 50. Through such a structure, the groove of the receiver 50 may be exposed at the center of the head part 30.

The head part 30 may include a first region 31 configured to irradiate light and transmit vibration, a second region 33 configured to transmit electrical stimulation and vibration, a third region 35 configured to irradiate light and transmit vibration, a fourth region 37 configured to transmit electrical stimulation and vibration, and a fifth region 39 configured to irradiate light and transmit vibration.

In one embodiment, the area of the first region 31 may be smaller than the area of the second region 33, the area of the second region 33 may be smaller than the area of the third region 35, the area of the third region 35 may be smaller than the area of the fourth region 37, and the area of the fourth region 37 may be larger than the area of the fifth region 39.

In one embodiment, the first region 31 may have a rim shape surrounding the outside of the groove of the receiver 50. Specifically, the first region 31 may have a rim shape surrounding the outside of a second groove 59 of the receiver 50. In one embodiment, the first region 31 may have a circular rim shape. However, it is not limited thereto and may be formed in various rim shapes such as a triangular rim, a quadrangular rim, an elliptical rim, and the like.

In one embodiment, the second region 33 may have a rim shape surrounding the outside of the first region 31. In one embodiment, the second region 33 may have a circular rim shape. However, it is not limited thereto and may be formed in various rim shapes such as a triangular rim, a quadrangular rim, an elliptical rim, and the like.

In one embodiment, the third region 35 may have a rim shape surrounding the outside of the second region 33. In one embodiment, the third region 35 may have a circular rim shape. However, it is not limited thereto and may be formed in various rim shapes such as a triangular rim, a quadrangular rim, an elliptical rim, and the like.

In one embodiment, the fourth region 37 may have a rim shape surrounding the outside of the third region 35. In one embodiment, the fourth region 37 may have a circular rim shape. However, it is not limited thereto and may be formed in various rim shapes such as a triangular rim, a quadrangular rim, an elliptical rim, and the like.

In one embodiment, the fifth region 39 may have a rim shape surrounding the outside of the fourth region 37. In one embodiment, the fifth region 39 may have a circular rim shape. However, it is not limited thereto and may be formed in various rim shapes such as a triangular rim, a quadrangular rim, an elliptical rim, and the like.

When the first region 31 to the fifth region 39 are circular rims, the diameter of the second region 33 may be larger than the diameter of the first region 31, the diameter of the third region 35 may be larger than the diameter of the second region 33, the diameter of the fourth region 37 may be larger than the diameter of the third region 35, and the diameter of the fifth region 39 may be larger than the diameter of the fourth region 37.

In the first direction (X-axis direction), the width of the first region 31 may be smaller than the width of the second region 33, the width of the second region 33 may be larger than the width of the third region 35, the width of the third region 35 may be smaller than the width of the fourth region 37, and the width of the fourth region 37 may be larger than the width of the fifth region 39.

In one embodiment, the head part 30 may include a first layer part 130 and a second layer part 230. The first layer part 130 may be seated and coupled to the second layer part 230. For example, the first layer part 130 may be coupled to the lower surface of the second layer part 230.

The first layer part 130 may be an electrode for transmitting electricity and may be made of a SUS material. In some embodiments, the first layer part 130 may be made of a SUS material, but a portion in contact with the user's skin may be coated with a thermoplastic synthetic resin.

In one embodiment, the first layer part 130 may include a first electrode 131 and a second electrode 133. The first electrode 131 and the second electrode 133 may be formed in a circular rim shape, but are not limited thereto.

A first coupling part 135 may be disposed on the first electrode 131, and the first coupling part 135 may have a bar (BAR) shape extending from the top of the first electrode 131 in a direction opposite to the second direction (Y-axis direction). A plurality of first coupling parts 135 may be disposed spaced apart.

A second coupling part 137 may be disposed on the second electrode 133, and the second coupling part 137 may have a bar (BAR) shape extending from the top of the second electrode 133 in a direction opposite to the second direction (Y-axis direction). A plurality of second coupling parts 137 may be disposed spaced apart.

The diameter of the first electrode 131 may be smaller than the diameter of the second electrode 133, and the area of the first electrode 131 may be smaller than the area of the second electrode 133.

In one embodiment, the second layer part 230 is a frame of the head part 30, and the second layer part 230 may be coupled to the lower portion of the main body part 11. The second layer part 230 includes a through-hole in the center, and the groove of the receiver 50 disposed inside the main body part 11 can be exposed to the outside through the through-hole. For example, the through-hole of the second layer part 230 and the groove of the receiver 50 may overlap in the second direction (Y-axis direction).

An LED module and a vibration motor may be disposed inside the second layer part 230.

The lower surface of the second layer part 230 may be made of a transparent or translucent material and may output light output from the LED module to the outside. In addition, the second layer part 230 may vibrate by a vibration motor. In some embodiments, the LED module and the vibration motor may be disposed on the upper portion of the second layer part 230. The vibration motor may be turned ON when the first electrode 131 and the second electrode 133 come into contact with the user's skin. For example, the vibration motor may be driven in an electrode contact detection method that detects a flow of current and drives when the first electrode 131 and the second electrode 133 contact the user's skin at the same time. However, it is not limited thereto, and the vibration motor may be driven in a capacitance-based touch detection method that operates by detecting a change in capacitance, or may be driven in a force sensor method using a sensor whose resistance value changes according to pressure.

In one embodiment, the second layer part 230 may include a first seating part 231 and a second seating part 232. The first seating part 231 and the second seating part 232 may be disposed on the lower surface of the second layer part 230. The first seating part 231 and the second seating part 232 may be formed in a circular rim shape, but are not limited thereto.

The first seating part 231 and the second seating part 232 may be disposed spaced apart in the first direction (X-axis direction), the through-hole and the first seating part 231 may be disposed spaced apart, and the outer surface of the second seating part 232 and the second layer part may be spaced apart. The first seating part 231 and the second seating part 232 may be disposed spaced apart in a radial direction of the body 10.

The spaced region between the through-hole and the first seating part 231 may correspond to the aforementioned first region 31, the first seating part 231 may correspond to the second region 33, the spaced region between the first seating part 231 and the second seating part 232 may correspond to the third region 35, the second seating part 232 may correspond to the fourth region 37, and the spaced region between the second seating part 232 and the outer surface of the second layer part may correspond to the fifth region 39.

The first seating part 231 and the second seating part 232 may be disposed in a form protruding in a downward direction from the lower surface of the second layer part 230. Accordingly, a step may be formed on the lower surface of the second layer part 230. In addition, the protruding heights of the first seating part 231 and the second seating part 232 may be the same.

A first coupling hole 238 and a second coupling hole 239 may be disposed on the upper surface of the second layer part 230. The first coupling hole 238 and the second coupling hole 239 may be formed passing through the second layer part 230.

The first coupling hole 238 may be located on a boundary surface between the second region 33 and the third region 35, and a plurality of first coupling holes 238 may be disposed along the boundary surface between the second region 33 and the third region 35.

The second coupling hole 239 may be located on a boundary surface between the third region 35 and the fourth region 37, and a plurality of second coupling holes 239 may be disposed along the boundary surface between the third region 35 and the fourth region 37.

In one embodiment, the first electrode 131 is seated on the first seating part 231, and the first coupling part 135 may be inserted and fixed into the first coupling hole 238. In addition, the first coupling part 135 may pass through the first coupling hole 238 and be connected to the upper portion of the second layer part 230, and the first coupling part 135 may receive a pulse signal supplied by the control circuit and transmit it to the first electrode 131. The control circuit may convert power received from the battery into a short and strong pulse signal as described above. Such a pulse signal is transmitted to the first electrode 131 to allow a pulse current to flow to the user's skin.

In one embodiment, the second electrode 133 is seated on the second seating part 232, and the second coupling part 137 may be inserted and fixed into the second coupling hole 239. In addition, the second coupling part 137 may pass through the second coupling hole 239 and be connected to the upper portion of the second layer part 230, and the second coupling part 137 may receive a pulse signal supplied by the control circuit and transmit it to the second electrode 133. The control circuit may convert power received from the battery into a short and strong pulse signal as described above. Such a pulse signal is transmitted to the second electrode 133 to allow a pulse current to flow to the user's skin.

In one embodiment, the light output from the LED module may be blocked from being output to the outside by the first electrode 131 and the second electrode 133. However, it is not limited thereto, and it may have a structure in which a part of the light output from the LED module is output to the outside.

The second layer part 230 may include a rib 234, a fitting groove 235, and a third coupling part 237. The rib 234, the fitting groove 235, and the third coupling part 237 may be disposed on the upper surface of the second layer part 230.

The rib 234 may be in a cylindrical protruding form and may have a circular groove formed therein. However, it is not limited thereto. A plurality of ribs 234 may be disposed on the upper surface of the second layer part 230.

The fitting groove 235 may be a semi-circular recessed portion. However, it is not limited thereto. A plurality of fitting grooves 235 may be disposed along the outside of the upper surface of the second layer part 230.

The third coupling part 237 may be in a bar (BAR) shape extending from the top of the second layer part 230 in a direction opposite to the second direction (Y-axis direction). A plurality of third coupling parts 237 may be disposed along the outside of the upper surface of the second layer part 230.

In one embodiment, the receiver 50 may be disposed in the internal space of the body 10. The receiver 50 may include a receiving body 51 including a receiving space therein, a fixing part 53 protruding outside the receiving body 51, a rib coupling groove 55 passing through the fixing part 53, and a third coupling groove 57 disposed at an end of the fixing part 53.

The receiving body 51 may include a groove which is a receiving space. In one embodiment, the groove may include a first groove 58 and a second groove 59. The first groove 58 and the second groove 59 may each be formed in a cylindrical shape, but are not limited thereto. The first groove 58 and the second groove 59 may be sequentially positioned in the second direction (Y-axis direction).

The first groove 58 and the second groove 59 may overlap in the second direction (Y-axis direction), and a diameter of the first groove 58 in the first direction (X-axis direction) may be smaller than a diameter of the second groove 59 in the first direction (X-axis direction). Due to such a difference in diameter between the first groove 58 and the second groove 59, a step may be formed in the boundary region between the first groove 58 and the second groove 59, and a cosmetic container or the like can be inserted and fixed through such a step.

A plurality of fixing parts 53 may be disposed along the outer surface of the receiving body 51. In addition, the rib 234 of the second layer part 230 may be coupled to the rib coupling groove 55 passing through the fixing part 53. The rib 234 of the second layer part 230 may be inserted and fixed into the rib coupling groove 55. In another embodiment, the rib 234 of the second layer part 230 is disposed to correspond to the rib coupling groove 55, and the rib 234 of the second layer part 230 may be fixed to the rib coupling groove 55 through a separate coupling means such as a screw.

The third coupling part 237 of the second layer part 230 may be inserted and fixed into the third coupling groove 57 disposed at the end of the fixing part 53.

In addition, a plurality of protrusions may be disposed on the inner surface of the receiving body 51, and the fitting groove 235 of the second layer part 230 may be seated and fixed to the protrusions.

While the embodiments of the present invention have been described above with reference to the accompanying drawings, those of ordinary skill in the art to which the present invention pertains will understand that the present invention can be implemented in other specific forms without changing its technical spirit or essential features. Therefore, it should be understood that the embodiments described above are illustrative in all respects and not restrictive.

What is claimed is:

1. A skin device comprising:
a body; and
a head part disposed at one end of the body,
wherein the head part is configured to transmit electrical stimulation, light irradiation, and vibration to a user's skin,
wherein the body includes a main body part having a storage space capable of accommodating components therein, and a button disposed on an outer surface of the main body part to control power,
wherein the skin device further comprises a receiver disposed in an internal space of the body,
wherein the receiver includes a receiving body having a receiving space therein,
wherein the receiving body forms a groove,
wherein the head part includes a first layer part and a second layer part,
wherein the first layer part is seated and coupled to the second layer part,
wherein the first layer part includes electrodes configured to transmit electricity,
wherein the electrodes include a first electrode and a second electrode,
wherein each of the first electrode and the second electrode has a circular rim shape,
wherein a diameter of the first electrode is smaller than a diameter of the second electrode, and
wherein the second layer part is coupled to a lower portion of the main body part and has a through-hole, and the groove is exposed through the through-hole.

2. The skin device of claim 1,
wherein the body further includes a grip, and
wherein the grip is formed on the outer surface of the main body part and is disposed in a form recessed inward from the outer surface of the main body part.

3. The skin device of claim 1,
wherein the head part includes:
a first region configured to irradiate light and transmit vibration;
a second region configured to transmit electrical stimulation and vibration;
a third region configured to irradiate light and transmit vibration;
a fourth region configured to transmit electrical stimulation and vibration; and
a fifth region configured to irradiate light and transmit vibration.

4. The skin device of claim 3,
wherein an area of the first region is smaller than an area of the second region,
wherein the area of the second region is smaller than an area of the third region,
wherein the area of the third region is smaller than an area of the fourth region, and
wherein the area of the fourth region is larger than an area of the fifth region.

5. The skin device of claim 4,
wherein the first region has a circular rim shape surrounding an outside of the groove,
wherein the second region has a circular rim shape surrounding an outside of the first region,
wherein the third region has a circular rim shape surrounding an outside of the second region,
wherein the fourth region has a circular rim shape surrounding an outside of the third region, and
wherein the fifth region has a circular rim shape surrounding an outside of the fourth region.

6. The skin device of claim 1,
wherein the second layer part is configured to output light to the outside and transmit vibration.

7. The skin device of claim 1,
wherein the second layer part includes a first seating part and a second seating part, and
wherein each of the first seating part and the second seating part has a circular rim shape.

8. The skin device of claim 7,
wherein the first seating part and the second seating part are disposed spaced apart from each other in a first direction.

9. The skin device of claim 7,
wherein the first electrode is seated on the first seating part, and the second electrode is seated on the second seating part.

10. The skin device of claim 1,
wherein the first electrode and the second electrode are configured to transmit electrical stimulation to a user's skin.

11. The skin device of claim 1,
wherein each of the first electrode and the second electrode is made of a stainless steel material.

12. The skin device of claim 1,
wherein a lower surface of the second layer part is made of a transparent or translucent material.

13. The skin device of claim 8,
wherein the groove includes a first groove and a second groove,
the first groove and the second groove are sequentially positioned in a second direction crossing the first direction, and
a diameter of the first groove in the first direction is smaller than a diameter of the second groove in the first direction.

14. The skin device of claim 1,
wherein the skin device is configured such that a cosmetic container is coupled to and separated from the receiver.
